(19) United States
(12) Patent Application Publication
(10) Pub. No.: US 2025/0014722 A1
(43) Pub. Date: Jan. 9, 2025

(54) PERSONALIZED IMAGE MODIFICATION FOR CLINICAL SETTINGS

(71) Applicant: Align Technology, Inc., San Jose, CA (US)

(72) Inventors: Doruk Cetin, Zürich (CH); Alain Souchet, Mulhouse (FR); Eric Paul Meyer, Zürich (CH); Nico Benjamin Huber, Zug (CH); Petar Stamenkovic, Zürich (CH); Majed El Helou, Zürich (CH); Fabio Zünd, Bern (CH)

(21) Appl. No.: 18/761,665

(22) Filed: Jul. 2, 2024

Related U.S. Application Data
(60) Provisional application No. 63/525,409, filed on Jul. 7, 2023.

(51) Int. Cl. G16H 30/40; G06T 7/10; G06T 7/70; G06T 7/90; G06T 11/60; G06V 40/16; G16H 30/20

(52) U.S. Cl. CPC G16H 30/40; G06T 7/10; G06T 7/70; G06T 7/90; G06T 11/60; G06V 40/174; G06V 40/178; G16H 30/20

(57) ABSTRACT

A method comprises receiving a first image comprising first clinical information and first non-clinical information of a first patient and receiving a second image comprising second clinical information and second non-clinical information of the first patient or a second patient. The method further comprises generating a third image of the first patient based on the first image and the second image, wherein one of a) the third image is generated based on the first clinical information and the second non-clinical information such that the third image resembles a combination of the first clinical information and the second non-clinical information or b) the third image is generated based on the second clinical information and the first non-clinical information such that the third image resembles a combination of the second clinical information and the first non-clinical information.

### RELATED APPLICATIONS

This patent application claims the benefit under 35 U.S.C. § 119(e) of U.S. Provisional Application No. 63/525,409, filed Jul. 7, 2023, which is incorporated by reference herein.

### TECHNICAL FIELD

Embodiments of the present invention relate to the field of dentistry and, in particular, to the generation of patient images that are personalized to a dental patient.

### BACKGROUND

When a dentist or orthodontist is engaging with current and/or potential patients, it is often helpful to show those patients images of before and after treatments of previous patients with similar malocclusions who have undergone successful treatment. However, often those previous patients look very different from the current patient. For example, the current patient may be a young woman and the previous patient may be an old man with a beard. Such differences can make it difficult for the current or potential patient to properly visualize how they might look after successful treatment due to these differences. The more differences there are between the current patient and the prior patients whose images are shown, the more distracting those differences can become, which detract from the current patient's ability to visualize themselves with similar corrected teeth.

### SUMMARY

Various example implementations are summarized. These example implementations are merely for illustration and should not be construed as limiting.

In a 1^{st} implementation, a method comprises: receiving a first image comprising first clinical information and first non-clinical information of a first patient; receiving a second image comprising second clinical information and second non-clinical information of the first patient or a second patient; and generating a third image of the first patient based on the first image and the second image, wherein one of a) the third image is generated based on the first clinical information and the second non-clinical information such that the third image resembles a combination of the first clinical information and the second non-clinical information or b) the third image is generated based on the second clinical information and the first non-clinical information such that the third image resembles a combination of the second clinical information and the first non-clinical information.

A 2^{nd} implementation may further extend the 1^{st} implementation. In the 2^{nd} implementation, the first image was generated at a first time, and wherein the second image is of the first patient and was generated at a second time.

A 3^{rd} implementation may further extend the 2^{nd} implementation. In the 3^{rd} implementation, the first time corresponds to a first patient visit of the first patient and the second time corresponds to a second patient visit of the first patient.

A 4^{th} implementation may further extend the 2^{nd} implementation. In the 4^{th} implementation, the first clinical information comprises a first condition of a dentition of the first patient, and wherein the second clinical information comprises a second condition of the dentition of the first patient.

A 5^{th} implementation may further extend the 4^{th} implementation. In the 5^{th} implementation, the first condition of the dentition of the first patient corresponds to pre-treatment or a first stage of treatment, and wherein the second condition of the dentition of the first patient corresponds to a second stage of treatment.

A 6^{th} implementation may further extend any of the 1^{st} through 5^{th} implementations. In the 6^{th} implementation, the first image comprises a post-treatment image of the first patient after orthodontic treatment, wherein the second image comprises a pre-treatment image of the second patient, wherein the first clinical information of the first patient comprises a dentition of the first patient after the orthodontic treatment, and wherein the second non-clinical information comprises an appearance of the second patient other than a dentition of the second patient.

A 7^{th} implementation may further extend any of the 1^{st} through 6^{th} implementations. In the 7^{th} implementation, the first non-clinical information comprises a first appearance of the first patient, and wherein the second non-clinical information comprises a second appearance of the first patient or the second patient.

An 8^{th} implementation may further extend the 7^{th} implementation. In the 8^{th} implementation, the first appearance comprises at least one of a first pose, a first facial angle, a first makeup application, a first gender, a first facial expression, first clothing, first lighting conditions, a first background, a first haircut, a first weight, a first hair color, a first skin tone, a first age, a first facial structure, or first wearable accessories; and the second appearance comprises at least one of a second pose, a second facial angle, a second makeup application, a second gender, a second facial expression, second clothing, second lighting conditions, a second background, a second haircut, a second weight, a second hair color, a second skin tone, a second age, a second facial structure, or second wearable accessories.

A 9^{th} implementation may further extend the 7^{th} or 8^{th} implementation. In the 9^{th} implementation, the first image is a first facial image, wherein the second image is a second facial image, wherein the first appearance comprises a first facial appearance, and wherein the second appearance comprises a second facial appearance.

A 10^{th} implementation may further extend any of the 1^{st} through 9^{th} implementations. In the 10^{th} implementation, generating the third image comprises: processing the first image and the second image using one or more trained machine learning models that extract at least one of the first clinical information or the first non-clinical information from the first image and at least one of the second clinical information or the second non-clinical information from the second image, and use the extracted information to generate the third image.

An 11^{th} implementation may further extend the 10^{th} implementation. In the 11^{th} implementation, the one or more trained machine learning models comprise a generative model.

A 12^{th} implementation may further extend the 10^{th} or 11^{th} implementation. In the 12^{th} implementation, the one or more trained machine learning models comprise a plurality of machine learning models each trained to generate a different feature for the third image and an additional machine learning model trained to process outputs of the plurality of machine learning models to output a photorealistic combination of the outputs of the plurality of machine learning models.

A 13^{th} implementation may further extend any of the 1^{st} through 12^{th} implementations. In the 13^{th} implementation, the third image comprises a photorealistic and clinically relevant synthetic image showing the first patient with specified changes in an appearance of the first patient attributable to the second clinical information of the second patient.

A 14^{th} implementation may further extend any of the 1^{st} through 13^{th} implementations. In the 14^{th} implementation, the first non-clinical information and the second non-clinical information each comprises a plurality of properties, the method further comprising: receiving selection of at least one of a) one or more of the plurality of properties to use from the first non-clinical information or b) one or more of the plurality of properties to use from the second non-clinical information in generation of the third image, wherein the third image is generated in accordance with the selection.

A 15^{th} implementation may further extend any of the 1^{st} through 14^{th} implementations. In the 15^{th} implementation, the method further comprises: segmenting at least one of the first image or the second image into a plurality of features; and using segmentation information determined from the segmenting in the generating of the third image.

A 16^{th} implementation may further extend the 15^{th} implementation. In the 16^{th} implementation, segmenting at least one of the first image or the second image into the plurality of features comprises processing at least one of the first image or the second image by a trained machine learning model that outputs the segmentation information.

A 17^{th} implementation may further extend any of the 1^{st} through 16^{th} implementations. In the 17^{th} implementation, the method further comprises: receiving a fourth image comprising third clinical information and third non-clinical information of the first patient, the second patient, or a third patient; wherein the third image is further generated from the third non-clinical information.

An 18^{th} implementation may further extend any of the 1^{st} through 17^{th} implementations. In the 18^{th} implementation, the second image is of the first patient, the method further comprising: receiving a temporal series of images of the first patient, each image in the temporal series of images comprising additional clinical information and additional non-clinical information of the first patient; and for each respective image in the temporal series of images, generating a modified version of the image comprising the first non-clinical information from the first image and the additional clinical information from the respective image.

A 19^{th} implementation may further extend any of the 1^{st} through 18^{th} implementations. In the 19^{th} implementation, the method further comprises: receiving an input selecting values of one or more properties of non-clinical information to apply for the third image, wherein the values of the one or more properties of the non-clinical information do not correspond to properties of the first non-clinical information or the second non-clinical information; wherein the selected values of the one or more properties of the non-clinical information are reflected in the third image.

A 20^{th} implementation may further extend the 19^{th} implementation. In the 206^{th} implementation, the one or more selected values of the one or more properties comprise at least one of a selected age, a selected weight, or a selected illumination condition.

A 21^{st} implementation may further extend any of the 1^{st} through 20^{th} implementations. In the 21st implementation, the first image is a frame of a first video and the second image is a frame of a second video, and the third image is a frame of a third video, the method further comprising: receiving one or more additional frames of the first video comprising the first clinical information and the first non-clinical information of the first patient; receiving one or more additional frames of the second video comprising the second clinical information and the second non-clinical information of the first patient or the second patient; and generating one or more additional frames of the third video of the first patient based on the one or more additional frames of the first video and the one or more additional frames of the second video.

A 22^{nd} implementation may further extend any of the 1^{st} through 20^{th} implementations. In the 22^{nd} implementation, the first image is a frame of a first video and the third image is a frame of a third video, the method further comprising: receiving one or more additional frames of the first video comprising the first clinical information and the first non-clinical information of the first patient; and generating one or more additional frames of the third video of the first patient based on the one or more additional frames of the first video and the second image.

In a 23^{rd} implementation, a method comprises: receiving a first image or video comprising first clinical information and first non-clinical information of a first patient; receiving a second image or video comprising second clinical information and second non-clinical information of the first patient or a second patient; and generating a third video of the first patient based on the first image or video and the second image or video, wherein one of a) the third video is generated based on the first clinical information and the second non-clinical information such that the third video resembles a combination of the first clinical information and the second non-clinical information or b) the third video is generated based on the second clinical information and the first non-clinical information such that the third video resembles a combination of the second clinical information and the first non-clinical information.

A 24^{th} implementation may further extend any of the 1^{st} through 23^{rd} implementations. In the 24^{th} implementation, a computing device comprises a memory and a processing device operatively coupled to the memory, wherein the processing device is to perform the method of any of the 1^{st} through 23^{rd} implementations.

A 25^{th} implementation may further extend any of the 1^{st} through 23^{rd} implementations. In the 25^{th} implementation, a computer readable storage medium comprises instructions that, when executed by a processing device, cause the processing device to perform the method of any of the 1^{st} through 23^{rd} implementations.

### BRIEF DESCRIPTION OF THE DRAWINGS

Embodiments of the present invention are illustrated by way of example, and not by way of limitation, in the figures of the accompanying drawings.

FIG. 1 illustrates workflows for training one or more machine learning models to perform image generation and applying the trained machine learning models to images to generate new synthetic images, in accordance with embodiments of the present invention.

FIG. 2 illustrates a flow diagram for a method of generating a synthetic image using non-clinical information from a first image of a patient and clinical information from a second image of the patient, in accordance with an embodiment.

FIG. 3 illustrates generation of a synthetic image of a patient by an image generator based on combined properties of a first and second image of the patient, in accordance with an embodiment.

FIG. 4 illustrates a flow diagram for a method of generating a synthetic image using non-clinical information from a first image of a first patient and clinical information from a second image of a second patient, in accordance with an embodiment.

FIG. 5 illustrates generation of a synthetic image of a first patient by an image generator based on combined properties of a first image of the patient and a second image of a different patient, in accordance with an embodiment.

FIG. 6 illustrates a block diagram of an example computing device, in accordance with embodiments of the present invention.

### DETAILED DESCRIPTION

Described herein are methods and systems for generating synthetic images based on non-clinical information from one or more first images and clinical information from one or more second images. As used herein, clinical information may refer to information that is based on, relevant to, and/or characterized by an observable and diagnosable symptom or condition of a patient. In one embodiment, clinical information comprises visual information of a patient's teeth or dentition. As used herein, non-clinical information may refer to information that is not based on, relevant to, or characterized by the observable and diagnosable symptom or condition of the patient that is reflected in the clinical information. In one embodiment, non-clinical information includes information for every region of a patient's face other than the region depicting the patient's teeth or dentition. The images may be two-dimensional (2D) images, three-dimensional (3D) images, frames of 2D video, frames of a 3D video, x-ray images, cone-beam computed tomography (CBCT) scans, and so on.

In embodiments, first and second images are images of one or more patients or other persons before or after orthodontic treatment or another medical treatment that will affect the patient's appearance. In the first and second images, which may be images of a patient smiling and showing his or her teeth, the clinical information may include information on the patient's teeth or dentition.

In one embodiment, a first image of a patient may be captured at a first time (e.g., prior to treatment of the patient) and a second image may be captured at a second time (e.g., after treatment of the patient has begun). The treatment may be, for example, orthodontic treatment. In the first and second images, which may be images of the patient smiling and showing his or her teeth, the clinical information may include information on the patient's teeth or dentition. The patient's dentition may be different in the first and second images due to the orthodontic treatment. However, it may be difficult for the patient to easily compare how his or her teeth have improved between the first and second image due to differences in non-clinical information between the images. For example, the patient may have different clothing, may be wearing different accessories, have a different hair styles and/or hair color, have a different amount of facial hair, be in a different pose (e.g., different rotation of patient), have a different facial angle relative to the camera (e.g., be taken from different angled views), have a different facial expression, be in different lighting conditions, and so on in the different images. In embodiments, generated images may be from any camera angle relative to an imaged subject (e.g., may have any facial angles). For example, generated images may include frontal images, lateral images, or images with any other facial angle. In embodiments, an image generator may receive the first and second images of the patient, and may generate a new image that preserves the clinical information (e.g., dentition) of the patient from the second image while preserving the non-clinical information (e.g., clothing, worn accessories, hair style and/or hair color, amount of facial hair, pose, facial angle, facial expression, lighting conditions, etc.) from the first image. The patient may then be shown the first image and the new image side-by-side, and the only or main differences between the images may be the clinical differences (e.g., the differences in the dentition). This makes it much easier for the patient to easily identify the improvement in their clinical condition between the first and second images.

Data from images of different patients or persons may also be combined in embodiments. In one embodiment, an image of a first patient may be captured after treatment of the patient (e.g., after orthodontic treatment). Additionally, a second image may be captured of a second patient or potential patient who has not yet undergone treatment. It can be beneficial for the second patient to see how they might be affected by the treatment. To help the second patient to visualize how they would like after treatment, an image generator may use clinical information from the first image of the first patient and non-clinical information from the second image of the second patient to generate a new image that shows the second patient but with a post-treatment condition (e.g., with post-treatment dentition that could be expected after orthodontic treatment is performed). The first patient may have had a similar malocclusion or other condition that is shared by the second patient. Simply showing the second patient images of the first patient may not enable the second patient to visualize how they might look with similar treatment. Additionally, a doctor may not have authorization to show images of the first patient to the second patient. By processing the first image and the second image by an image generator, the image generator may generate a synthetic image that combines the post-treatment condition (e.g., dentition) of the first patient with image data of the second patient to show the second patient with the post-treatment condition without actually showing the first image of the first patient to the second patient. Therefore, the first patient's privacy may be maintained, and at the same time the second patient is shown an image of themselves with the post-treatment condition. This makes it much easier for the second patient to visualize what they will look like if the treatment is performed on them, leaving little or nothing to the second patient's imagination. Accordingly, embodiments enable a doctor to showcase previous treatment results of prior patients in a personalized manner for new patients.

In embodiments, one or more machine learning models are trained to perform image generation from an input of two or more images. The one or more machine learning models may be, for example, generative models such as generators of generative adversarial networks (GANs). Other types of generative models that may be used include diffusion models, gaussian splatting models, and so on. In some embodiments, multiple generators are used together, where some generators may generate images of particular features of a person, and an additional generator may generate an image of the person based on the generated images of the particular features of the person output by the other generators. In some embodiments, one or more trained machine learning models are trained to perform segmentation of input images and output segmentation information. The segmentation information of the two or more images input into the one or more generators may be used together with the image data of the two or more images to generate a new synthetic image that combines properties of the input images in embodiments.

In an example, multiple deep learning models may be arranged in sequence so that a first deep learning model performs segmentation of input images, a set of second deep learning models each generate an image or feature vector of a feature of a person in the input image or images, and a final deep learning model generates a synthetic image of the person based on the generated images of the features of the person.

Certain embodiments are described herein with reference to images of patients before and after treatment. However, it should be understood that embodiments also apply to other persons, animals, etc. who may not be patients. Accordingly, any reference to patients also applies to other subject, which may be, for example, persons or animals.

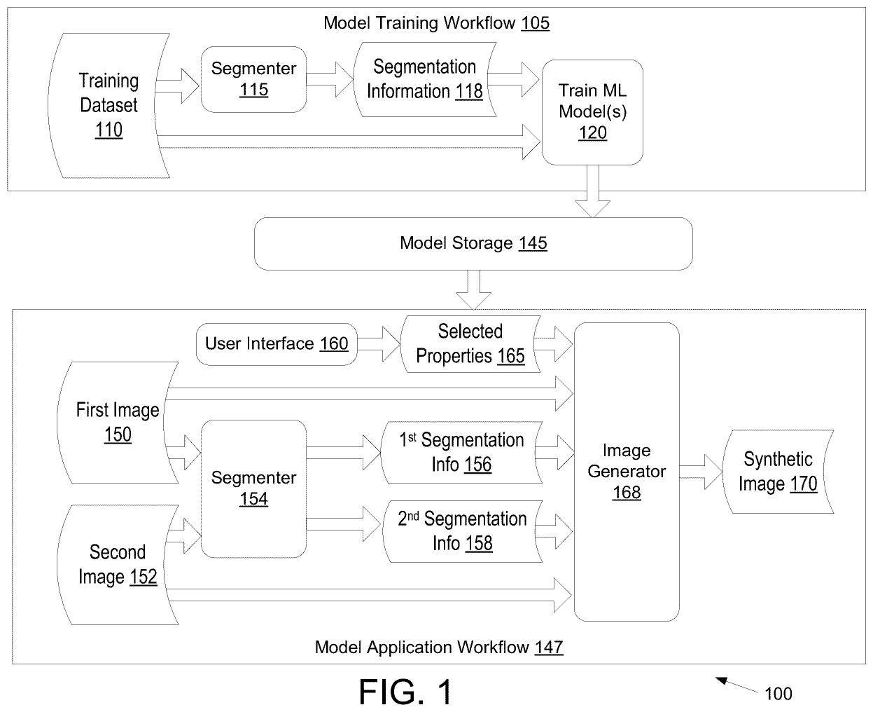

Referring now to the figures, FIG. 1 illustrates workflows 100 for training one or more machine learning models to perform image generation and applying the trained machine learning models to images to generate new synthetic images, in accordance with embodiments of the present invention. The illustrated workflows include a model training workflow 105 and a model application workflow 147. The model training workflow 105 is to train one or more machine learning models (e.g., deep learning models, generative models, etc.) to perform one or more image segmentation tasks and/or image generation tasks (e.g., for images of smiling persons showing their teeth). The model application workflow 147 is to apply the one or more trained machine learning models to segment input images and/or generate synthetic images from the input images.

One type of machine learning model that may be used is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

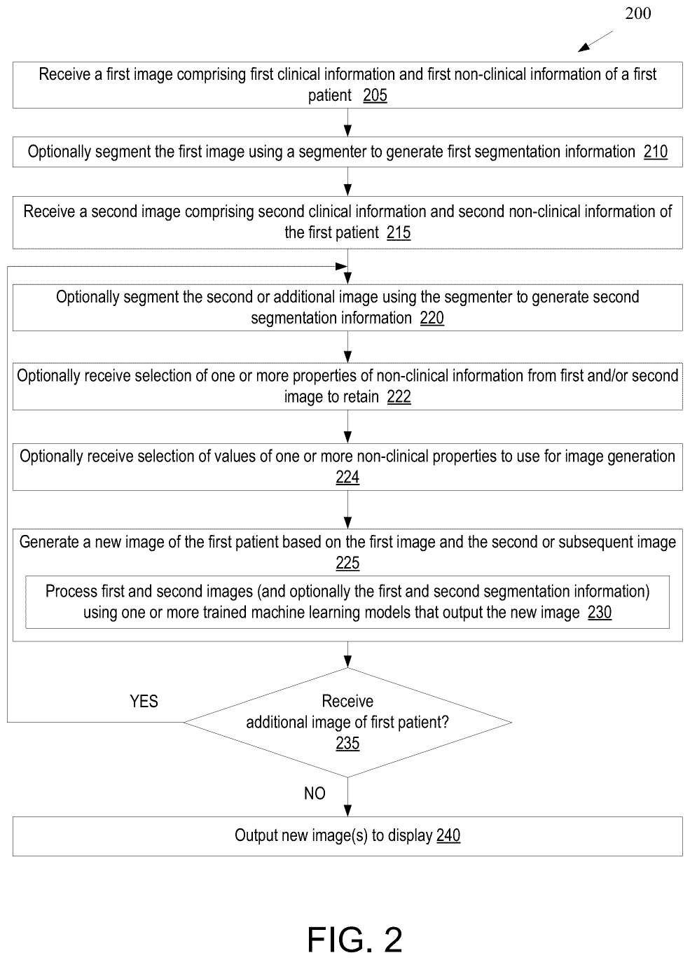

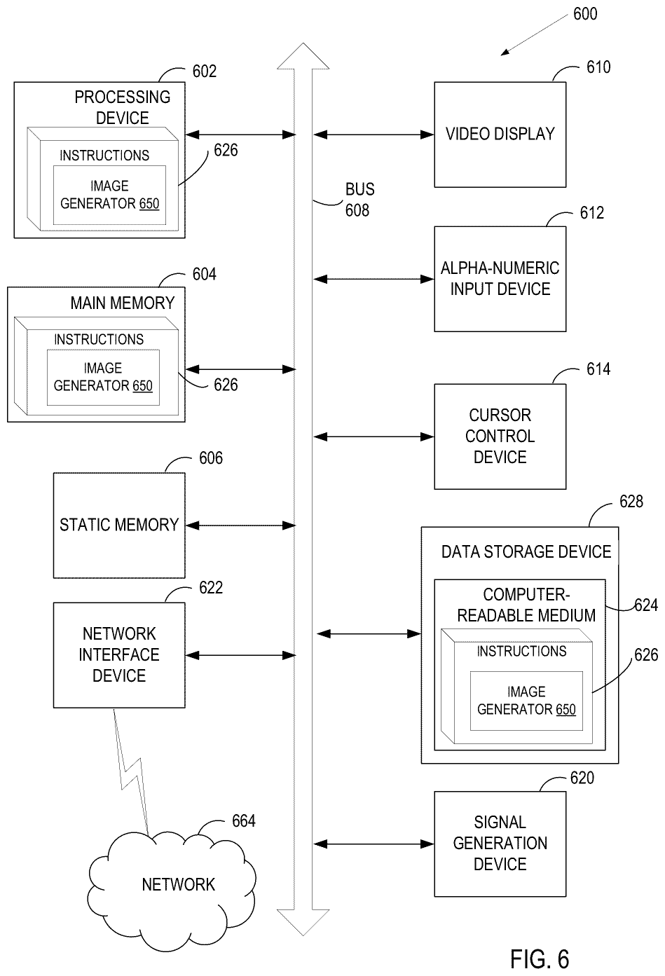

The model training workflow 105 and the model application workflow 147 may be performed by processing logic executed by a processor of a computing device. These workflows 105, 147 may be implemented, for example, by one or more modules executing on a processing device 602 of computing device 600 shown in FIG. 6. Additionally FIGS. 2-5 below describe example operations and/or methods associated with generating synthetic images that combine image properties from multiple input images (e.g., by applying one or more trained machine learning models to two or more input images). The operations and/or methods described with reference to FIGS. 2-5 may be performed by processing logic that may comprise hardware (e.g., circuitry, dedicated logic, programmable logic, microcode, etc.), software (e.g., instructions run on a processing device to perform hardware simulation), or a combination thereof. These methods and/or operations may be implemented, for example, by one or more machine learning modules executing on a processing device 602 of computing device 600 shown in FIG. 6.

For the model training workflow 105, a training dataset 110 containing hundreds, thousands, tens of thousands, hundreds of thousands or more images (e.g., images that include faces of patients showing their teeth) may be provided. The images may include front view images of faces, side view images of faces, front view and side view images of faces, images of faces from any facial angle, occlusal views of mouths and/or other images, and so on. The images may include two-dimensional (2D) images in one embodiment. Alternatively, or additionally, the images may include three-dimensional (3D) images. The images may additionally or alternatively include videos, x-ray images, CBCT scans, ultrasound images, and so on.

In some embodiments, some or all of the images may be labeled with segmentation information. The segmentation information may identify facial features such as nose, eyes, teeth, lips, ears, hair, etc., information identifying clothing, worn accessories, lighting conditions, gender, age, weight, one or more region of clinical information, one or more region of non-clinical information, and so on.

In some embodiments, images in the training dataset 110 are processed by a segmenter 115 that segments the images into multiple different features, and that outputs segmentation information for the images. The segmenter may be or include, for example, a trained machine learning model such as a convolutional neural network (CNN) trained to classify pixels or regions of input images into different classes. This can include performing point-level classification (e.g., pixel-level classification or voxel-level classification) of different types of features and/or objects of subjects of images. The different features and/or objects may include, for example, an area of interest containing clinical information and other areas not containing clinical information. The other areas not containing clinical information may include features and/or objects that may be identified, such as teeth, gingiva, lips, nose, eyes, ears, hair, clothing, eyewear, wearable accessories, lighting conditions, pose, facial angle, gender, age, and so on. The segmenter may output one or more masks, each of which may have a same resolution as an input image. The mask or masks may include a different identifier for each identified feature or object, and may assign the identifiers on a pixel-level or patch-level basis. In one embodiment, different masks are generated for one or more different classes of features and/or objects. In one embodiment, a single mask or map includes segmentation information for all identified classes of features and/or objects. Some types of features identified by segmenter 115 may be image-wide features, such as age, weight, lighting conditions, and so on. Other types of features are location-specific features and are represented in one or more masks.

In some embodiments, the segmenter performs one or more image processing and/or computer vision techniques or operations to extract segmentation information from images. Such image processing and/or computer vision techniques may or may not include the use of trained machine learning models. Accordingly, in some embodiments the segmenter 115 does not include a machine learning model. Some examples of image processing and/or computer vision techniques that may be performed by segmenter 115 includes determining a color distribution of skin tone, determining an illumination of an image, determining a hair color, and so on.

Images from the training dataset 110 and segmentation information 118 for those images may be used to train one or more machine learning models to generate synthetic images at block 120. Training a machine learning model may include first initializing the machine learning model. The machine learning model that is initialized may be a deep learning model such as an artificial neural network. Initialization of the artificial neural network may include selecting starting parameters for the neural network. The solution to a non-convex optimization algorithm depends at least in part on the initial parameters, and so the initialization parameters should be chosen appropriately.

In one embodiment, a generative adversarial network (GAN) is used for one or more machine learning models. A GAN is a class of artificial intelligence system that uses two artificial neural networks contesting with each other in a zero-sum game framework. The GAN includes a first artificial neural network (a generator or generative network) that generates candidates and a second artificial neural network (a discriminator or discriminative network) that evaluates the generated candidates. The generative network learns to map from a latent space to a particular data distribution of interest (a data distribution of changes to input images that are indistinguishable from photographs to the human eye), while the discriminative network discriminates between instances from a training dataset and candidates produced by the generator. The generative network's training objective is to increase the error rate of the discriminative network (e.g., to fool the discriminator network by producing novel synthesized instances that appear to have come from the training dataset). The generative network and the discriminator network are co-trained, and the generative network learns to generate images that are increasingly more difficult for the discriminative network to distinguish from real images (from the training dataset) while the discriminative network at the same time learns to be better able to distinguish between synthesized images and images from the training dataset. The two networks of the GAN are trained once they reach equilibrium. The GAN may include a generator network that generates artificial intraoral images and a discriminator network that segments the artificial intraoral images.

In one embodiment, one or more machine learning model is a conditional generative adversarial (cGAN) network, such as pix2pix. These networks not only learn the mapping from input image to output image, but also learn a loss function to train this mapping. GANs are generative models that learn a mapping from random noise vector z to output image y, G: z→y. In contrast, conditional GANs learn a mapping from observed image x and random noise vector z, to y, G: {x, z}→y. The generator G is trained to produce outputs that cannot be distinguished from “real” images by an adversarially trained discriminator, D, which is trained to do as well as possible at detecting the generator's “fakes”.

In one embodiment, one or more machine learning model is a style generative adversarial network (StyleGAN). StyleGAN is an extension to a GAN architecture to give control over disentangled style properties of generated images. In at least one embodiment, a generative network is a generative adversarial network (GAN) that includes a generator model and a discriminator model, where a generator model includes use of a mapping network to map points in latent space to an intermediate latent space, includes use of the intermediate latent space to control style at each point in the generator model, and uses introduction of noise as a source of variation at one or more points in the generator model. A resulting generator model is capable not only of generating impressively photorealistic high-quality synthetic images, but also offers control over a style of generated images at different levels of detail through varying style vectors and noise. Each style vector may correspond to a parameter or feature of clinical information or a parameter or feature of non-clinical information. For example, there may be one style vector for clinical information and one style vector for non-clinical information in embodiments. In another example, there may be a different style vector for each of multiple different features or parameters of non-clinical information. For example, there may be a style vector for hair style, hair color, facial hair, skin color, pose, facial angle, facial expression, age, weight, wearable accessories, lighting conditions, makeup application, gender, clothing, background, facial structure, and so on. In at least one embodiment, a generator starts from a learned constant input and adjusts a “style” of an image at each convolution layer based on a latent code, therefore directly controlling a strength of image features at different scales.

In at least one embodiment, a StyleGAN generator uses two sources of randomness used to generate a synthetic image: a standalone mapping network and noise layers, in addition to a starting point from latent space. An output from a mapping network is a vector that defines styles that is integrated at each point in a generator model via a layer called adaptive instance normalization. Use of this style vector gives control over style of a generated image. In at least one embodiment, stochastic variation is introduced through noise added at each point in a generator model. Noise may be added to entire feature maps that allow a model to interpret a style in a fine-grained, per-pixel manner. This per-block incorporation of style vector and noise allows each block to localize both an interpretation of style and a stochastic variation to a given level of detail.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many inputs in the training dataset yields a network that can produce correct output when presented with inputs that are different than the ones present in the training dataset. In high-dimensional settings, such as large images, this generalization is achieved when a sufficiently large and diverse training dataset is made available.

Training may be performed by inputting sets of two or more of the images into the machine learning model at a time. Each input may include data from multiple images, and optionally segmentation information 118 for those images. One or more generator model may generate one or more synthetic images from the input images and output the one or more synthetic images. One or more discriminator models may then determine whether or not the generated synthetic images are real or synthetic and output a determination. The determination may then be used to update the generator model(s) and/or the discriminator model(s).

An artificial neural network includes an input layer that consists of values in a data point (e.g., intensity values and/or height values of pixels in a height map). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer. An output layer of a generator model may output a synthetic image. An output layer of a discriminator model may output a determination as to whether or not an image is synthetic.

Processing logic adjusts weights of one or more nodes in the machine learning model(s) based on the output of the discriminator model(s). The output of the discriminator model(s) be used to determine an error term or delta may for each node in the generator model(s) and/or discriminator model(s). Based on this error, the artificial neural networks adjust one or more of their parameters for one or more of their nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the generator model(s) are able to generate images that are indistinguishable or close to indistinguishable from real images by the discriminator network(s), the training of the generator models may be complete.

Once one or more trained ML models are generated, they may be stored in model storage 145, and may be added to a treatment planning and/or visualization application. The treatment planning and/or visualization application may then use the one or more trained ML models as well as additional processing logic to generate photorealistic and clinically accurate synthetic images of patients in embodiments.

In one embodiment, model application workflow 147 includes one or more trained machine learning models that function as a segmenter 154 and an image generator 168. These logics may be implemented as separate machine learning models or as a single combined machine learning model in embodiments. For example, segmenter 154 and image generator 168 may share one or more layers of a deep neural network. However, each of these logics may include distinct higher level layers of the deep neural network that are trained to generate different types of outputs.

A doctor may capture an image of a current patient, which may correspond to first image 150 or second image 152. The doctor may have also captured a previous image of the patient and/or may have previously captured an image of a different patient (or may have access to images of other patients that may be used), which may correspond to first image 150 or second image 152. The images 150, 152 may be two-dimensional (2D) images, three-dimensional (3D) images, frames of 2D video, frames of a 3D video, x-ray images, cone-beam computed tomography (CBCT) scans, and so on. The first image 150 and second image 152 may be processed by segmenter 154, which may correspond to segmenter 115 in embodiments. The segmenter 154 may generate first segmentation information 156 of the first image 150 and may generate second segmentation information 158 of the second image 152.

The application that applies the model application workflow 147 (e.g., treatment planning and/or visualization software) may include a user interface 160 (e.g., such as a graphical user interface (GUI)). The user interface may provide options for selection of one or more properties of the first image 150 and/or second image 152 for inclusion in a generated synthetic image. For example, via the user interface 160 a user may select to retain the facial hair, hair color, worn accessories, pose, facial angle, facial expression, lighting conditions, age, weight, and so on from the first or second images 150, 152. In some embodiments, the user interface 160 receives the first and second images 150, 152 and/or first and/or second segmentation information 156, 158. The images 150, 152 and/or segmentation information 156, 158 may be output to a display. A user may then use a mouse pointer or touch screen, for example, to select (e.g., click on) regions in one or both of the images. Regions that are selected in an image may be retained in a generated synthetic image in embodiments. In some embodiments, the user interface 160 provides a menu (e.g., a drop down menu) providing different features of clinical and/or non-clinical information from the first image and/or features of clinical and/or non-clinical information from the second image. The menu may include generic graphics for the respective features, may not include graphics for the respective features, or may include custom graphics for the respective features as determined from the images 150, 152 and/or segmentation information 156, 158. From the menu a user may select which features to retain from the first image and/or which features to retain from the second image.

The user interface 160 may additionally provide options that enable a user to select values of one or more properties (e.g., of non-clinical information) to be included in generated synthetic images. The selected values for the properties may not correspond to values of the properties from either of the first image 150 or the second image 152. For example, the properties may include age, facial hair, hair color, weight, worn accessories, clothing, pose, facial angle, facial expression, or any of the other properties discussed elsewhere herein. The user interface 160 may include, for example, a slider associated with one or more features. A user may move a slider position of such a slider to a desired location, which may be associated with a particular value of the property for that feature. For example, if the feature or property is age, then a slider may be provided, and a position on the slider may be adjusted to adjust the apparent age of a subject of a synthetic image generated from the first and second images 150, 152.

In an example, a patient may be curious to see how they will look with speculative changes, such as changes to their age, hair, weight, tattoos, and so on. The patient may be curious, for example, about how medical treatment results would look not only for their current appearance, but also with respect to expected or speculative changes to their appearance. These speculative changes may be provided as inputs, and such inputs may be used together with the images to generate the new image that reflects the selected speculative changes.

Image generator 168 receives the first and second images 150, 152, and optionally receives first segmentation information 156, second segmentation information 158 and/or selected properties to retain from the first and/or second images 150, 152 and/or values of one or more properties to apply for a generated image. The image generator 168 receives these inputs and processes them to generate a synthetic image 170. The synthetic image may retain the clinical information from one of the images 150, 152 and may retain the non-clinical information from another of the images 150, 152. In some embodiments, some non-clinical information and clinical information from one of the images is retained and other non-clinical information of the second image is retained in the synthetic image.

Image generator 168 may generate a new synthetic image 170 that retains first selected or predetermined information (e.g., clinical and/or non-clinical information) from the first image and that retains second selected or predetermined information (e.g., clinical and/or non-clinical information) from the second image. In some embodiments, via user interface 160 a user may select not to retain certain non-clinical information from any of the input images. For example, a user may select not to use the hair style from the first or second images. The user interface 160 may provide different options for a particular property or parameter of non-clinical information, which a user may select from. That particular property or parameter may then be used in generation of the synthetic image 170. In some instances, a user may choose to have the particular property or parameter of non-clinical information to be randomized, in which case the image generator 168 may randomly generate that information for the synthetic image 170. In some embodiments, one or more aspects or features of a patient from an image are anonymized in generation of the synthetic image. In one embodiment, anonymization of the one or more aspects or features may be performed as set forth in U.S. application Ser. No. 18/140,196, filed Apr. 27, 2023, which is incorporated by reference herein in its entirety.

In some embodiments, via user interface 160 a user may select weights to apply to one or more features (e.g., non-clinical features or parameters) from one or more image for generation of the synthetic image 170. For example, a user may select to apply a 70% weight for the facial hair in the first image and to apply a 30% weight for the facial hair in the second image. The synthetic image 170 may then include a weighted combination of the particular feature. In some embodiments, the weights may be selectable via a slider presented in the user interface. For example, a user may select a property, and then adjust a slider associated with that property.

As indicated above, clinical information include information that is based on, relevant to, and/or characterized by an observable and diagnosable symptom or condition of a patient (e.g., visual information of a patient's teeth or dentition). Non-clinical information may include information that is not based on, relevant to, or characterized by the observable and diagnosable symptom or condition of the patient that is reflected in the clinical information (e.g., may include information for every region of a patient's face other than the region depicting the patient's teeth or dentition).

In some embodiments, treatment data corresponding with a patient may be useful in identifying clinical information (e.g., in identifying a region of interest (ROI) containing clinical information). As used herein, treatment data can include virtually any information relating to a potential clinical intervention associated with the patient. Examples of treatments include orthodontic treatment, prosthodontic treatment, plastic surgery, cosmetic dental treatment, weight gain, weight loss, and so on. In some examples, treatment data can be used to automatically determine one or more ROI containing clinical information in images. In some embodiments, treatment data (e.g., an indication of a type of treatment to be performed) is input into segmenter 115 and/or image generator 168. Segmenter 115 and/or image generator 168 may then use the provided treatment data when determining which areas of the first image and which areas of the second image to retain or mimic in generation of a new image.

In at least one embodiment, image generator 168 is trained to generate medical images of humans that include clinical information on human anatomy and non-clinical information of humans. In other embodiments, image generator 168 is trained to generate medical images of animal anatomy, other types of medical images, images of streets, images of buildings, images of manufactured products, images of nature scenes, images of human faces, and/or other types of images.

FIG. 2 illustrates a flow diagram for a method 200 of generating a synthetic image using non-clinical information from a first image of a patient and clinical information from a second image of the patient, in accordance with an embodiment. In embodiments, method 200 is performed using model application workflow 147 of FIG. 1. At block 205 of method 200, processing logic receives a first image comprising first clinical information and first non-clinical information of a patient. The first image may be, for example, a pre-treatment image of a face of the patient in which the patient is smiling and showing their teeth.

At block 210, the first image is optionally segmented into a plurality of features and/or parts using a segmenter (e.g., a trained machine learning model trained to perform image segmentation). The segmenter may output segmentation information, which may include one or more map or mask assigning different object identifiers to different pixels in the first image. In an example, the segmenter may segment an image of a face showing teeth into a clinical information segment comprising pixels corresponding to the patient's teeth and non-clinical information segment comprising pixels not corresponding to the patient's teeth. In another example, the segmenter may segment an image of a face showing teeth into a clinical information segment comprising pixels corresponding to the patient's teeth and multiple non-clinical information segments, each comprising a different non-clinical feature, such as a nose, hair, ears, eyes, eyebrows, face, skin, chin, a wearable accessory, a background, clothing, facial hair, and so on. The segmenter may also determine one or more classifications of the image, such as an age of the patient, a weight of the patient, a skin color of the patient, lighting in the first image, a facial expression in the first image, a pose in the first image, a facial angle in the first image, a makeup application in the first image, etc. that may apply to the image as a whole rather than a particular region of the image.

At block 215, processing logic receives a second image of the patient, where the second image comprises second clinical information and second non-clinical information. The second image may have been generated after a medical treatment has begun, such as after orthodontic treatment has begun. The clinical information of the second image may correspond to a change in a condition of the patient caused by the medical treatment. For example, if the medical treatment was orthodontic treatment, the clinical information in the second image may include updated dentition of the patient (e.g., in which a malocclusion has been corrected or partially corrected). One of more aspects of the patient may have changed between the first image and the second image. For example, the patient may be wearing different clothing, have a different hair style, have a different hair color, have a different amount of facial hair, have a different makeup application, have a different expression, have a different post, have a different background, have different wearable accessories, have different lighting conditions, and so on. All of these differences constitute changes in non-clinical information between the first image and the second image. Such changes in non-clinical information can be distracting and make it more difficult it notice the differences in the clinical information between the first and second images.

At block 220, the second image is optionally segmented into a plurality of features and/or parts using the segmenter (e.g., a trained machine learning model trained to perform image segmentation). The segmenter may output segmentation information, which may include one or more map or mask assigning different object identifiers to different pixels in the second image. In an example, the segmenter may segment the second image of a face showing updated teeth into a clinical information segment comprising pixels corresponding to the patient's updated teeth and non-clinical information segment comprising pixels not corresponding to the patient's updated teeth. In another example, the segmenter may segment the updated image of a face showing updated teeth into a clinical information segment comprising pixels corresponding to the patient's updated teeth and multiple non-clinical information segments, each comprising a different non-clinical feature, such as a nose, hair, ears, eyes, eyebrows, face, skin, chin, a wearable accessory, a background, clothing, facial hair, and so on. The segmenter may also determine one or more classifications of the second image, such as an age of the patient, a weight of the patient, a skin color of the patient, lighting in the second image, a facial expression in the second image, a pose in the second image, a facial angle in the second image, a makeup application in the second image, etc. that may apply to the second image as a whole rather than a particular region of the image.

At block 222, processing logic optionally receives a selection of one or more properties of the non-clinical information and/or clinical information of the first image and/or second image to retain. A user interface may be provided, and via the user interface a user may select one or more regions or features of the first image and/or of the second image to retain (e.g., by clicking on those regions or features in a display of the first and/or second images, or by selecting the regions or features from a dropdown menu). Alternatively, selection of which features and/or regions to retain from the first image and from the second image may be determined automatically without user input. For example, default settings may be provided to use all of the non-clinical information from the first image and use all of the clinical information from the second image.

At block 224, processing logic optionally receives a selection of values for one or more properties of non-clinical information and/or clinical information to apply in generating a new image. The selected values may or may not correspond to values reflected in the first and/or second images. Properties of non-clinical information for which values may be selected may include age, skin color, gender, weight, amount of facial hair, hairstyle, wearable accessories, and so on. For example, a slider may be presented in the user interface for adjusting a patient age. Using the slider, a user may select an advanced age that is older than the patient's age in either of the first or second image.

At block 225, processing logic generates a new image of the patient based on the first image and the second image. In some embodiments, processing logic further receives the first segmentation information and/or the second segmentation information, and uses the first and/or second segmentation information along with the first and second images to generate the new image. In some embodiments, no segmentation information is provided. In some embodiments, first selection information indicating a selection of features/properties of the first and/or second images to retain is also received. In some embodiments, second selection information indicating values of one or more features/properties to use in generation of the new image are received. Any or all of the above indicated information may be received in embodiments.

In some embodiments, at block 230 processing logic inputs the first and second images (and optionally the first and/or second segmentation information and/or the first and/or second selection information) into a trained machine learning model, which may be a generative model. In some embodiments, the trained machine learning model includes one or more layers that perform segmentation of the received images, and segments the first image into third segmentation information and the second image into fourth segmentation information. This may be performed in some embodiments whether or not segmentation information is provided as an input to the machine learning model. The machine learning model may include multiple generative models trained to generate specific features of a new image. These generative models may receive the first and/or second image and the first, second, third and/or fourth segmentation information (and optionally the first and/or second selection information), and may generate a particular feature or property for the new image. The machine learning model may further include an additional generative model that receives the outputs of the other generative models (e.g., synthetic images of particular parameters or features, such as images of teeth, lips, ears, eyes, hair, background, etc.), and may generate the new model based on the outputs of the other generative models. The new image may be clinically accurate, and accurately depict the clinical information from the second image. At the same time, the new image may be photorealistic and may show the patient in a manner such that the patient looks essentially the same in the new image as they did in the first image, except for the differences in the clinical information between the first and second image. For example, the patient may have had a malocclusion, a first hair color, first hair length, glasses, a dress, red lipstick, a first pose, a first facial angle, a first facial expression, and earrings in the first image. The patient may have lacked the malocclusion, had a second hair color, had a second hair length, not worn glasses, had on overalls, not worn lipstick, had a second pose, had a second facial angle, had a second facial expression, and not had on earrings in the second image. The new image may show the patient's dentition without the malocclusion, and may further show the first hair color, the first hair length, the glasses, the dress, the red lipstick, the first pose, the first facial angle, the first facial expression, and the earrings. Accordingly, the patient and/or doctor may compare the first image to the new image to easily identify the differences in the patient's dentition caused by their orthodontic treatment.

Method 200 may be performed on a sequence of images in addition to a single image. In some embodiments, method 200 is performed on a video. For example, a first image or video of the patient may have been generated at a first time (e.g., pre-treatment), and a second image or video may have been generated at a second time (e.g., at an intermediate stage of treatment or after completion of treatment). Method 200 may be performed with any combination of a first image and a second image, a first image and a second video, a first video and a second image, or a first video and a second video in embodiments. Operations 205-230 or 252-230 may be performed for each frame of the first video and/or second video, as appropriate. Accordingly, block 225 may output multiple new images, where each new image may be a frame of a new video. These frames may be put together to form a new video. The new video may show the patient moving and/or from multiple different viewpoints, expressions, poses, facial angles, etc. to provide a better depiction of their post-treatment condition. A first video may be presented to a user alongside a new synthetic video. The two videos may be shown in-sync, so that the patient has the same position, pose, facial angle, facial expression, etc. in each video at any given time. This enables the user to view the clinical differences caused by the treatment from different perspectives, across different facial expressions, across different posts, and so on.

In one embodiment, at block 235 processing logic determines whether an additional image has been received for the patient. If an additional image has been received, the method may return to block 220, and operations 220-225 may be repeated for the additional image. For example, a temporal series of images may have been generated of the patient. Each image in the temporal series of images may have been generated at a different time, and may reflect a different stage of treatment, different clothing, different hair styles, different poses, different facial angles, different facial expressions, and so on. The first image may be separately used together with each of the additional images to generate new versions of each of the additional images that retain the clinical information from those images but apply the non-clinical information from the first image. Alternatively, non-clinical information may be retained from any of the other images in the series. As long as the same non-clinical information (e.g., a specific set of features) is applied for each newly generated image (e.g., the new version of each image in the series), then each of the new images in the series will look very similar, and may differ by only the clinical information. Accordingly, changes to a patient's medical condition (e.g., changes to the patient's dentition) over a course of treatment may be easily viewed. In embodiments, a video may be generated based on the changes to the patient's medical condition over time. The video may show changing dentition, while the other non-clinical properties may not change or may change minimally over the course of the video.

If no additional images have been received, the method may continue to block 240. At block 240, processing logic outputs the new image, new images, new video, etc. to a display. The new image, images, video, etc. may be output together with the first image for comparison purposes in embodiments.

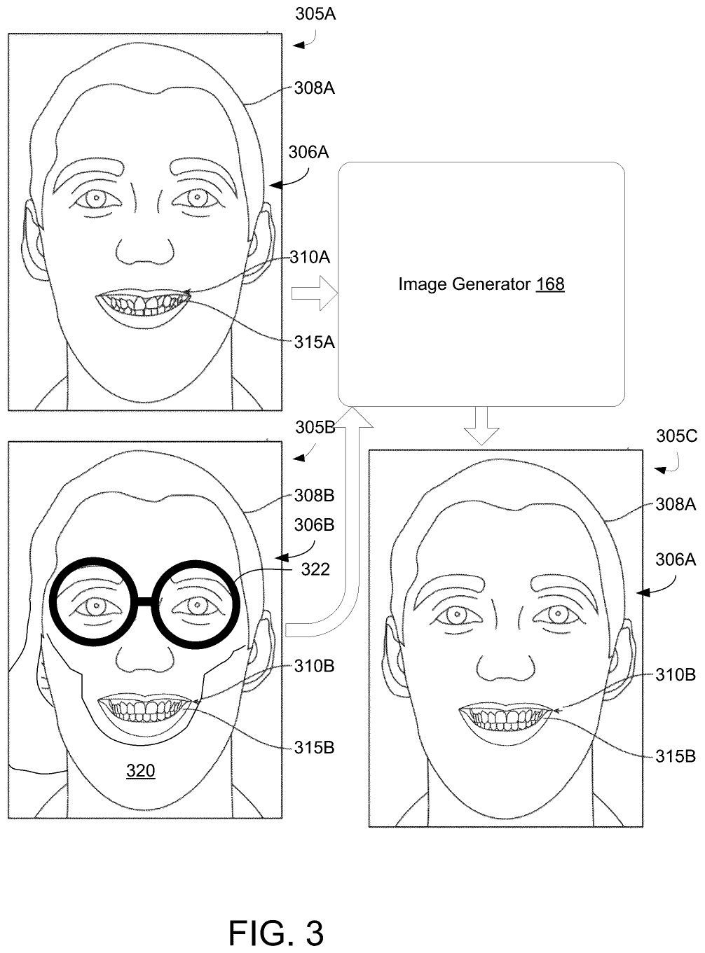

FIG. 3 illustrates generation of a synthetic image of a subject (e.g., a patient) by an image generator based on combined properties of a first and second image of the patient, in accordance with an embodiment. As shown, a first image 305A and a second image 305B are input into image generator 168. In the first image 305A, a subject has first clinical properties 310A including a first dentition 315A and first non-clinical properties 306A including first hair 308A, a lack of facial hair, and lack of glasses. In the second image 305B, the subject has second clinical properties 310B including a second dentition 315B and second non-clinical properties 306B including second hair 308B, facial hair 320, and glasses 322. As shown, first dentition 315A includes a malocclusion, while second dentition 315B lacks the malocclusion.

Image generator 168 generates new image 305C based on inputs of the first image 305A and second image 305B. In the new image 305C, the subject has second clinical properties 310B including second dentition 31BA that lacks the malocclusion and first non-clinical properties 306A including first hair 308A, no facial hair, and no glasses.

As set forth in FIGS. 2-3, embodiments can be used to generate new images of a particular subject (e.g., a particular patient) based on two images of that subject. Additionally, in embodiments more than two images of the subject may be used to generate a new image. If more than two images of the subject are used, then some properties of the subject may be retained from the first image, some properties of the subject may be retained from the second image, and some properties of the subject may be retained from the third and/or subsequent image. Which properties to retain from which images may be automatically determined or may be based on a received selection.

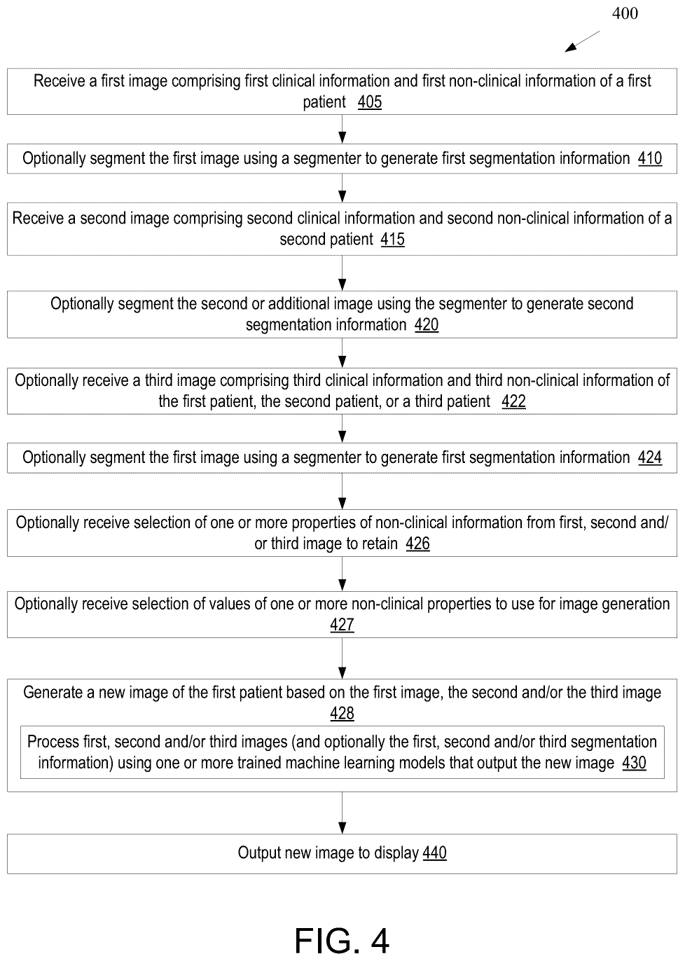

FIG. 4 illustrates a flow diagram for a method 400 of generating a synthetic image using non-clinical information from a first image of a first patient and clinical information from a second image of a second patient, in accordance with an embodiment. Method 400 may be applied, for example, to show a subject who has not yet undergone treatment what they might look like after treatment based on the outcome of a prior patient who underwent a successful treatment. In embodiments, method 400 is performed using model application workflow 147 of FIG. 1. At block 405 of method 400, processing logic receives a first image comprising first clinical information and first non-clinical information of a first subject. The first image may be, for example, a post-treatment image of a face of the first subject in which the first subject is smiling and showing their teeth. In one example, the first subject is a prior patient who has successfully undergone treatment, such as orthodontic treatment. The first subject may have originally had a condition (e.g., a malocclusion) that is similar to the condition of a second subject, who may be a current or potential patient of a doctor. The first clinical information of the first image may include, for example, the first subject's post-treatment dentition after orthodontic treatment, and may not include any malocclusion.

At block 410, the first image is optionally segmented into a plurality of features and/or parts using a segmenter (e.g., a trained machine learning model trained to perform image segmentation). The segmenter may output segmentation information, which may include one or more map or mask assigning different object identifiers to different pixels in the first image. In an example, the segmenter may segment the first image of a face showing post-treatment teeth into a clinical information segment comprising pixels corresponding to the first subject's teeth and non-clinical information segment comprising pixels not corresponding to the first subject's teeth. In another example, the segmenter may segment the image of a face showing the first subject's teeth into a clinical information segment comprising pixels corresponding to the first subject's teeth and multiple non-clinical information segments, each comprising a different non-clinical feature, such as a nose, hair, ears, eyes, eyebrows, face, skin, chin, a wearable accessory, a background, clothing, facial hair, and so on. The segmenter may also determine one or more classifications of the first image, such as an age of the first subject, a weight of the first subject, a skin color of the first subject, lighting in the first image, a facial expression in the first image, a pose in the first image, a facial angle in the first image, a makeup application in the first image, etc. that may apply to the first image as a whole rather than a particular region of the image.

At block 415, processing logic receives a second image of the second subject, where the second image comprises second clinical information and second non-clinical information. The second image may have been generated of the second subject prior to performance of treatment. The clinical information of the second image may show one or more medical conditions to be treated, such as a malocclusion to be treated via orthodontic treatment.

Since the second subject is different from the first subject, they may have many differences. For example, the first and second subjects may be different gender, have a different facial structure, have a different hair type, have a different hair color, be wearing different clothing, have a different hair style, have a different amount of facial hair, have a different makeup application, have a different expression, have a different pose, have a different facial angle, have a different background, have different wearable accessories, have different lighting conditions, and so on. Such differences in non-clinical information can be distracting and make it difficult for the subject to imagine what they would look like if they were to undergo the same treatment as the first subject.

At block 420, the second image is optionally segmented into a plurality of features and/or parts using the segmenter (e.g., a trained machine learning model trained to perform image segmentation). The segmenter may output segmentation information, which may include one or more map or mask assigning different object identifiers to different pixels in the second image. In an example, the segmenter may segment the second image of a face showing pre-treatment teeth into a clinical information segment comprising pixels corresponding to the second subject's teeth and non-clinical information segment comprising pixels not corresponding to the second subject's teeth. In another example, the segmenter may segment the image of a face showing the second subject's teeth into a clinical information segment comprising pixels corresponding to the second subject's teeth and multiple non-clinical information segments, each comprising a different non-clinical feature, such as a nose, hair, ears, eyes, eyebrows, face, skin, chin, a wearable accessory, a background, clothing, facial hair, and so on. The segmenter may also determine one or more classifications of the second image, such as an age of the second subject, a weight of the second subject, a skin color of the second subject, lighting in the second image, a facial expression in the second image, a pose in the second image, a facial angle in the second image, a makeup application in the second image, etc. that may apply to the second image as a whole rather than a particular region of the image.

At block 422, processing logic optionally receives a third image of the first subject, the second subject, or a third subject, where the third image comprises third clinical information and third non-clinical information. At block 424, the third image is optionally segmented into a plurality of features and/or parts using the segmenter (e.g., a trained machine learning model trained to perform image segmentation).

At block 426, processing logic optionally receives a selection of one or more properties of the non-clinical information and/or clinical information of the first image, the second image and/or the third image to retain. A user interface may be provided, and via the user interface a user may select one or more regions or features of the first image, the second image and/or of the third image to retain (e.g., by clicking on those regions or features in a display of the first, second and/or third images, or by selecting the regions or features from a dropdown menu). Alternatively, selection of which features and/or regions to retain from the first image, the second image and/or the third image may be determined automatically without user input. For example, default settings may be provided to use all of the clinical information from the first image and use all of the non-clinical information from the second image.

At block 427, processing logic optionally receives a selection of values for one or more properties of non-clinical information and/or clinical information to apply in generating a new image. The selected values may or may not correspond to values reflected in the first and/or second images. Properties of non-clinical information for which values may be selected may include age, skin color, gender, weight, amount of facial hair, hairstyle, wearable accessories, and so on. For example, a slider may be presented in the user interface for adjusting a patient age. Using the slider, a user may select an advanced age that is older than the patient's age in either of the first or second image.

At block 428, processing logic generates a new image of the patient based on the first image, the second image and/or the third image. In some embodiments, processing logic further receives the first segmentation information, the second segmentation information and/or the third segmentation information, and uses the first, second and/or third segmentation information along with the first, second and/or third images to generate the new image. In some embodiments, no segmentation information is provided. In some embodiments, first selection information indicating a selection of features/properties of the first, second and/or third images to retain is also received. In some embodiments, second selection information indicating values of one or more features/properties to use in generation of the new image are received. Any or all of the above indicated information may be received in embodiments.

In some embodiments, at block 430 processing logic inputs the first, second and/or third images (and optionally the first, second and/or third segmentation information and/or the first and/or second selection information) into a trained machine learning model, which may be a generative model. In some embodiments, the trained machine learning model includes one or more layers that perform segmentation of the received images, and segments the first image into fourth segmentation information, the second image into fifth segmentation information and the third image into sixth segmentation information. This may be performed in some embodiments whether or not segmentation information is provided as an input to the machine learning model. The machine learning model may include multiple generative models trained to generate specific features of a new image. These generative models may receive the first, second and/or third image and the first, second, third, fourth, fifth and/or sixth segmentation information, and may generate a particular feature or property for the new image. The machine learning model may further include an additional generative model that receives the outputs of the other generative models (e.g., synthetic images of particular parameters or features, such as images of teeth, lips, ears, eyes, hair, background, etc.), and may generate the new image based on the outputs of the other generative models. The new image may be clinically accurate, and accurately depict the clinical information from the first image. At the same time, the new image may be photorealistic and may show the second subject in a manner such that the second subject looks essentially the same in the new image as they did in the second image, except for the differences in the clinical information between the first and second image. For example, the second subject may have a malocclusion, a first hair color, first hair length, glasses, a dress, red lipstick, a first pose, a first facial angle, a first facial expression, and earrings in the first image. The first subject may have lacked any malocclusion, had a second hair color, had a second hair length, not worn glasses, had on overalls, not worn lipstick, had a second pose, had a second facial angle, had a second facial expression, and not had on earrings in the second image. The new image may show the second subject with the first subject's dentition without the malocclusion, and may show the hair color, the hair length, the glasses, the dress, the red lipstick, the pose, the facial angle, the facial expression, and the earrings of the second subject reflected in the second image. Accordingly, the patient and/or doctor may compare the second image to the new image to easily identify the differences in the patient's dentition that can be expected if the second subject undergoes similar orthodontic treatment that the first subject underwent.

Method 400 may be performed on a sequence of images in addition to a single image. In some embodiments, method 400 is performed on a video. For example, a first image or video of the first subject may have been generated at a first time (e.g., pre-treatment), and a second image or video may have been generated of the second subject at a second time (e.g., at an intermediate stage of treatment or after completion of treatment). Method 400 may be performed with any combination of a first image and a second image, a first image and a second video, a first video and a second image, or a first video and a second video in embodiments. Operations 405-230 or 415-430 may be performed for each frame of the first video and/or second video, as appropriate. Accordingly, block 428 may output multiple new images, where each new image may be a frame of a new video. These frames may be put together to form a new video. The new video may show the second subject moving and/or from multiple different viewpoints, expressions, poses, facial angles, etc. to provide a better depiction of their anticipated post-treatment condition. A second video may be presented to a user alongside a new synthetic video. The two videos may be shown in-sync, so that the second subject has the same position, pose, facial angle, facial expression, etc. in each video at any given time. This enables the user to view the clinical differences caused by the proposed treatment from different perspectives, across different facial expressions, across different posts, and so on.

At block 440, processing logic outputs the new image, new images, new video, etc. to a display. The new image, images, video, etc. may be output together with the second image, video, etc. for comparison purposes in embodiments.

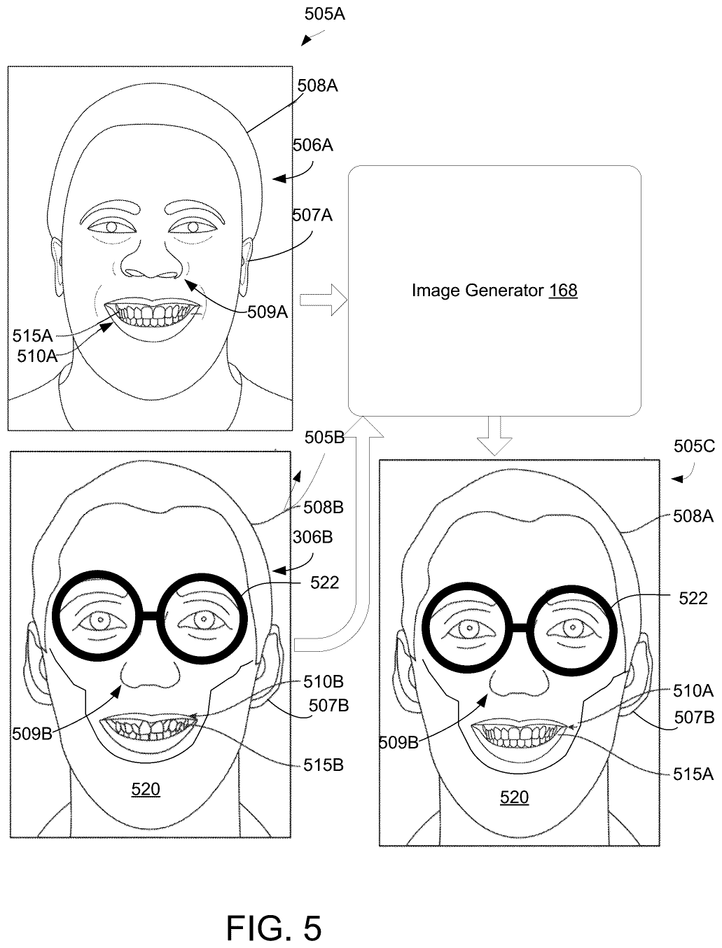

FIG. 5 illustrates generation of a synthetic image of a second subject by an image generator based on combined properties of a first image of a first subject and a second image of the second subject, in accordance with an embodiment. As shown, a first image 505A and a second image 505B are input into image generator 168. In the first image 505A, a first subject has first clinical properties 510A including a first dentition 515A and first non-clinical properties 506A including first hair 508A, first ears 507A, a first nose 509A, a lack of facial hair, and lack of glasses. In the second image 505B, the second subject has second clinical properties 510B including a second dentition 51BA and second non-clinical properties 506B including second hair 508B, facial hair 520, second nose 509B, second ears 507B, and glasses 522. As shown, first dentition 515A lacks a malocclusion, while second dentition 515B includes a malocclusion.

Image generator 168 generates new image 505C based on inputs of the first image 505A and second image 505B. In the new image 505C, the second subject has first clinical properties 510A including first dentition 515A that lacks the malocclusion and second non-clinical properties 506B including second hair 508B, facial hair 520, glasses 522, second nose 509B, second ears 507B, and so on.

FIG. 6 illustrates a diagrammatic representation of a machine in the example form of a computing device 600 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 600 includes a processing device 602, a main memory 604 (e.g., read-only memory (ROM), flash memory, dynamic random access memory (DRAM) such as synchronous DRAM (SDRAM), etc.), a static memory 606 (e.g., flash memory, static random access memory (SRAM), etc.), and a secondary memory (e.g., a data storage device 628), which communicate with each other via a bus 608.

Processing device 602 represents one or more general-purpose processors such as a microprocessor, central processing unit, or the like. More particularly, the processing device 602 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 602 may also be one or more special-purpose processing devices such as an application specific integrated circuit (ASIC), a field programmable gate array (FPGA), a digital signal processor (DSP), network processor, or the like. Processing device 602 is configured to execute the processing logic (instructions 626) for performing operations and steps discussed herein.

The computing device 600 may further include a network interface device 622 for communicating with a network 664. The computing device 600 also may include a video display unit 610 (e.g., a liquid crystal display (LCD) or a cathode ray tube (CRT)), an alphanumeric input device 612 (e.g., a keyboard), a cursor control device 614 (e.g., a mouse), and a signal generation device 620 (e.g., a speaker).

The data storage device 628 may include a machine-readable storage medium (or more specifically a non-transitory computer-readable storage medium) 624 on which is stored one or more sets of instructions 626 embodying any one or more of the methodologies or functions described herein, such as instructions for one or more machine learning modules and/or image generator 650. A non-transitory storage medium refers to a storage medium other than a carrier wave. The instructions 626 may also reside, completely or at least partially, within the main memory 604 and/or within the processing device 602 during execution thereof by the computer device 600, the main memory 604 and the processing device 602 also constituting computer-readable storage media.

The computer-readable storage medium 624 may also be used to store one or more machine learning modules and/or an image generator 650, which may perform the operations described herein above. The computer readable storage medium 624 may also store a software library containing methods for the one or more machine learning modules and/or image generator 650. While the computer-readable storage medium 624 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present invention. The term “computer-readable storage medium” shall accordingly be taken to include, but not be limited to, solid-state memories, and optical and magnetic media.

Various embodiments of the disclosure are discussed in detail above. While specific implementations are discussed, it should be understood that this is done for illustration purposes only. A person skilled in the relevant art will recognize that other components and configurations may be used without parting from the spirit and scope of the disclosure. Thus, the above description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Without intent to limit the scope of the disclosure, examples of instruments, apparatus, methods and their related results according to the embodiments of the present disclosure are given above.

It is to be understood that the above description is intended to be illustrative, and not restrictive. Many other embodiments will be apparent upon reading and understanding the above description. Although embodiments of the present invention have been described with reference to specific example embodiments, it will be recognized that the invention is not limited to the embodiments described, but can be practiced with modification and alteration within the spirit and scope of the appended claims. Accordingly, the specification and drawings are to be regarded in an illustrative sense rather than a restrictive sense. The scope of the invention should, therefore, be determined with reference to the appended claims, along with the full scope of equivalents to which such claims are entitled.

1. A system comprising:
a memory; and
a processing device operatively connected to the memory, wherein the processing device is to:
receive a first image comprising first clinical information and first non-clinical information of a first patient;
receive a second image comprising second clinical information and second non-clinical information of the first patient or a second patient; and
generate a third image of the first patient based on the first image and the second image, wherein one of a) the third image is generated based on the first clinical information and the second non-clinical information such that the third image resembles a combination of the first clinical information and the second non-clinical information or b) the third image is generated based on the second clinical information and the first non-clinical information such that the third image resembles a combination of the second clinical information and the first non-clinical information.

2. The system of claim 1, wherein the first image was generated at a first time, and wherein the second image is of the first patient and was generated at a second time.

3. The system of claim 2, wherein the first time corresponds to a first patient visit of the first patient and the second time corresponds to a second patient visit of the first patient.

4. The system of claim 2, wherein the first clinical information comprises a first condition of a dentition of the first patient, and wherein the second clinical information comprises a second condition of the dentition of the first patient.

5. The system of claim 4, wherein the first condition of the dentition of the first patient corresponds to pre-treatment or a first stage of treatment, and wherein the second condition of the dentition of the first patient corresponds to a second stage of treatment.

6. The system of claim 1, wherein the first image comprises a post-treatment image of the first patient after orthodontic treatment, wherein the second image comprises a pre-treatment image of the second patient, wherein the first clinical information of the first patient comprises a dentition of the first patient after the orthodontic treatment, and wherein the second non-clinical information comprises an appearance of the second patient other than a dentition of the second patient.

7. The system of claim 1, wherein the first non-clinical information comprises a first appearance of the first patient, and wherein the second non-clinical information comprises a second appearance of the first patient or the second patient.

8. The system of claim 7, wherein:
the first appearance comprises at least one of a first pose, a first facial angle, a first makeup application, a first gender, a first facial expression, first clothing, first lighting conditions, a first background, a first haircut, a first weight, a first hair color, a first skin tone, a first age, a first facial structure, or first wearable accessories; and
the second appearance comprises at least one of a second pose, a second facial angle, a second makeup application, a second gender, a second facial expression, second clothing, second lighting conditions, a second background, a second haircut, a second weight, a second hair color, a second skin tone, a second age, a second facial structure, or second wearable accessories.

9. The system of claim 7, wherein the first image is a first facial image, wherein the second image is a second facial image, wherein the first appearance comprises a first facial appearance, and wherein the second appearance comprises a second facial appearance.

10. The system of claim 1, wherein generating the third image comprises:
processing the first image and the second image using one or more trained machine learning models that extract at least one of the first clinical information or the first non-clinical information from the first image and at least one of the second clinical information or the second non-clinical information from the second image, and use the extracted information to generate the third image.

11. The system of claim 10, wherein the one or more trained machine learning models comprise a generative model.

12. The system of claim 10, wherein the one or more trained machine learning models comprise a plurality of machine learning models each trained to generate a different feature for the third image and an additional machine learning model trained to process outputs of the plurality of machine learning models to output a photorealistic combination of the outputs of the plurality of machine learning models.

13. The system of claim 1, wherein the third image comprises a photorealistic and clinically relevant synthetic image showing the first patient with specified changes in an appearance of the first patient attributable to the second clinical information of the second patient.

14. The system of claim 1, wherein the first non-clinical information and the second non-clinical information each comprises a plurality of properties, wherein the processing device is further to:
receive selection of at least one of a) one or more of the plurality of properties to use from the first non-clinical information or b) one or more of the plurality of properties to use from the second non-clinical information in generation of the third image, wherein the third image is generated in accordance with the selection.

15. The system of the claim 1, wherein the processing device is further to:
segment at least one of the first image or the second image into a plurality of features; and
use segmentation information determined from the segmenting in the generating of the third image.

16. The system of claim 15, wherein segmenting at least one of the first image or the second image into the plurality of features comprises processing at least one of the first image or the second image by a trained machine learning model that outputs the segmentation information.

17. The system of claim 1, wherein the processing device is further to:
receive a fourth image comprising third clinical information and third non-clinical information of the first patient, the second patient, or a third patient;
wherein the third image is further generated from the third non-clinical information.

18. The system of claim 1, wherein the second image is of the first patient, and wherein the processing device is further to:
receive a temporal series of images of the first patient, each image in the temporal series of images comprising additional clinical information and additional non-clinical information of the first patient; and
for each respective image in the temporal series of images, generate a modified version of the image comprising the first non-clinical information from the first image and the additional clinical information from the respective image.

19. The system of claim 1, wherein the processing device is further to:
receive an input selecting values of one or more properties of non-clinical information to apply for the third image, wherein the values of the one or more properties of the non-clinical information do not correspond to properties of the first non-clinical information or the second non-clinical information;
wherein the selected values of the one or more properties of the non-clinical information are reflected in the third image.

20. The system of claim 19, wherein the selected values of the one or more properties comprise at least one of a selected age, a selected weight, or a selected illumination condition.

21. The system of claim 1, wherein the first image is a frame of a first video and the second image is a frame of a second video, and the third image is a frame of a third video, and wherein the processing device is further to:
receive one or more additional frames of the first video comprising the first clinical information and the first non-clinical information of the first patient;
receive one or more additional frames of the second video comprising the second clinical information and the second non-clinical information of the first patient or the second patient; and
generate one or more additional frames of the third video of the first patient based on the one or more additional frames of the first video and the one or more additional frames of the second video.

22. The system of claim 1, wherein the first image is a frame of a first video and the third image is a frame of a third video, and wherein the processing device is further to:
receive one or more additional frames of the first video comprising the first clinical information and the first non-clinical information of the first patient; and
generate one or more additional frames of the third video of the first patient based on the one or more additional frames of the first video and the second image.

23. A non-transitory computer readable medium comprising instruction that, when executed by a processing device, cause the processing device to perform operations comprising:
receiving a first image or video comprising first clinical information and first non-clinical information of a first patient;
receiving a second image or video comprising second clinical information and second non-clinical information of the first patient or a second patient; and
generating a third video of the first patient based on the first image or video and the second image or video, wherein one of a) the third video is generated based on the first clinical information and the second non-clinical information such that the third video resembles a combination of the first clinical information and the second non-clinical information or b) the third video is generated based on the second clinical information and the first non-clinical information such that the third video resembles a combination of the second clinical information and the first non-clinical information.

24. A method comprising:
receiving a first image comprising first clinical information and first non-clinical information of a first patient;
receiving a second image comprising second clinical information and second non-clinical information of the first patient or a second patient; and
generating a third image of the first patient based on the first image and the second image, wherein one of a) the third image is generated based on the first clinical information and the second non-clinical information such that the third image resembles a combination of the first clinical information and the second non-clinical information or b) the third image is generated based on the second clinical information and the first non-clinical information such that the third image resembles a combination of the second clinical information and the first non-clinical information.
